(19) United States
(12) Patent Application Publication
(10) Pub. No.: US 2012/0041524 A1
(43) Pub. Date: Feb. 16, 2012

(54) Compositions for Providing Vitamin D Year Round and Uses Thereof

(76) Inventor: Michael F. Holick, Sudbury, MA (US)

(21) Appl. No.: 13/181,668

(22) Filed: Jul. 13, 2011

Related U.S. Application Data
(63) Continuation of application No. 12/153,435, filed on May 19, 2008, now abandoned.
Application No. 12/153,435 is a continuation of application No. 10/443,734, filed on May 22, 2003, now abandoned.
(60) Provisional application No. 60/382,194, filed on May 22, 2002.

(51) Int. Cl. A61N 5/06; A61K 31/592; A61P 3/00; A61P 43/00; A61P 17/06; A61P 19/08; A61P 13/12; A61P 5/18; A61K 31/575; A61P 19/10

(52) U.S. Cl. 607/94; 514/170; 514/167

(57) ABSTRACT

Methods for enhancing the ability of an individual, exposed to sunlight, to produce vitamin D via the skin. Pharmaceutical compositions comprising provitamin D and at least one of lumisterol and tachysterol and analogs and derivatives thereof are also disclosed.

### FIELD OF THE INVENTION

The invention is in the field of cosmetics and medicinal chemistry. In particular, the present invention relates to topical compositions which provide vitamin D and derivatives thereof throughout the year. The topical compositions of the invention allow a user in the high northern and southern latitudes to produce previtamin D on their skin even when exposed to low energy sunlight in the winter as well as in the morning and evening throughout the year. The compositions comprise provitamin D and at least one of tachysterol and lumisterol, and derivatives and analogs thereof, which photoisomerize to previtamin D, and analogs and derivatives thereof.

### BACKGROUND OF THE INVENTION

Vitamin D₃ is a derivative of provitamin D₃ (7-dehydrocholesterol), the immediate biological precursor of cholesterol. With adequate exposure to sunlight, dietary supplements are not normally required. Holick et al. in Braunwald et al., Harrison's Principles of Internal Medicine, 11th ed. McGraw-Hill (1987), pp. 1857-69. However, not all individuals are exposed to the adequate levels of sunlight, especially in the winter.

When skin is exposed to sunlight or artificial sources of ultraviolet (UV) radiation, the UV radiation penetrates the epidermis and causes a variety of biochemical reactions. Included in these reactions include the transformation of provitamin D₃ to previtamin D₃. The solar electromagnetic energy having wave-lengths between 290 and 315 nm is absorbed by provitamin D₃ resulting in its fragmentation to previtamin D₃. Although previtamin D₃ is biologically inert, it is thermally labile and spontaneously undergoes a temperature-dependent rearrangement to form the thermally stable vitamin D₃. After biosynthesis, vitamin D₃ is translocated from the epidermis into the circulation via a vitamin-D binding protein. Holick et al., Science 211:590-593 (1981); Holick et al. in Braunwald et al., Harrison's Principles of Internal Medicine, 11th ed., McGraw-Hill (1987), pp. 1857-69.

Factors that are frequently considered as affecting the cutaneous synthesis of vitamin D₃ include age, altitude, geographical location, time of day, seasonal changes and area of exposure to sunlight. Common to most of these factors is the availability of the requisite amount of ultraviolet radiation with energies between 290 and 315 nm which is necessary to convert provitamin D₃ to previtamin D₃. MacLaughlin et al., Science 216:1001-1003 (1982).

The availability of a vitamin D precursor (provitamin D₃) in the skin and its photo-induced transformation to previtamin D₃ and then to vitamin D₃ is an efficient physiological source of and mechanism for the replenishment of vitamin D₃. However, during the winter in the higher latitudes, sunlight does not contain enough high energy ultraviolet radiation to convert provitamin D₃ (7-dehydrocholesterol) in human skin to previtamin D₃. As a result, individuals in these latitudes cannot make vitamin D₃ in their skin, even when they are exposed to sunlight. Webb, Kline and Holick, J. Clin. Endocrin. Met. 67:373-378 (1988). The lack of adequate exposure to ultraviolet radiation gives rise to the possibility of serious vitamin D deficiency, a breakdown in blood calcium regulation with concomitant hypocalcemia and bone calcium wasting.

The availability of the vitamin D precursor in the skin and its photo-induced transformation to previtamin D₃, and then to vitamin D₃, is an efficient physiological source of, and mechanism for the replenishment of vitamin D₃. Previously, it was thought that the only method of producing previtamin D₃ in the skin was to transform provitamin D₃. This transformation requires sunlight or artificial UV light in the region of 290-315 nm. Therefore, in areas where the available light energy is below this range (wavelengths greater than 316 nm), the transformation does not occur to any significant extent. Kobayashi et al., J. Nutr. Sci. Vitaminol. 19:123 (1973).

It has been disclosed (Holick, M., Transactions of the Association of American Physicians, 42:54-63 (1979); Molecular Endocrinology; MacIntyre and Szelke, eds.; Elsevier/North Holland Biomedical Press (1979), pp. 301-308) that the topical application of hydroxylated metabolites of provitamin D compounds to the skin combined with UV phototherapy is a method for the sustained administration of vitamin D metabolites to patients who suffer vitamin D metabolic disorders. When the hydroxylated provitamins are applied and irradiated with ultraviolet radiation, they convert to hydroxylated previtamins which then thermally isomerize to the hydroxylated vitamin D. This work is also disclosed in Holick et al., New England Journal of Medicine 301:349-354 (1980) and U.S. Pat. No. 4,310,511 (Jan. 12, 1982).

Hungarian Patent No. 102,939 discloses cosmetic creams containing vitamin D precursors (such as ergosterol) which, when irradiated with ultraviolet rays, are transformed into vitamin D.

MacLaughlin et al., Science 216:1001-1003 (1982), disclose the synthesis of previtamin D₃ from provitamin D₃ in human skin and in an organic solvent after exposure to narrow-band radiation or simulated solar radiation. When human skin or an organic solvent containing provitamin D₃ were exposed to 295 nm radiation, up to 65% of the provitamin D₃ was converted to previtamin D₃. The authors further disclose that the optimum wavelength for the production of previtamin D₃ is between 295 nm and 300 nm.

Dauben et al., J. Am. Chem. Soc. 104:5780-5781 (1982); J. Am. Chem. Soc. 104:355-356 (1982), disclose the effect of wavelength on the photochemistry of provitamin D₃ and the effect of wavelength on the production of previtamin D₃. The authors found that when provitamin D₃ is exposed to light in the range of 254 nm, it is converted to a variety of photoproducts, the major portion being about 75% tachysterol. This mixture was then exposed to either 300 nm of light, broad-band 350 nm light or 355 nm light to give a build up of previtamin D₃. Dauben et al. conclude that if provitamin D₃ is first irradiated at 0° C. with 254 nm light to give a quasi photostationary state of provitamin D₃, previtamin D₃, tachysterol and lumisterol, and the mixture is thereafter irradiated (0° C.) with 350 nm light, a maximum of 83% previtamin D₃ is produced.

Malatesta et al., J. Amer. Chem. Soc. 103:6781-6783 (1981), disclose the effects of different UV wavelengths on the relative quantities of photoproducts produced from provitamin D₃.

Holick et al. disclose that the photochemical conversion of previtamin D₃ to lumisterol and tachysterol is the major factor that prevents vitamin D₃ intoxication after a single prolonged exposure to the sun. Holick et al., Science 211:590-592 (1981). The corollary to this finding is that lumisterol and tachysterol are two biologically inert products thought to be sloughed off the skin during the natural turnover of the epidermal cells.

Provitamin D₂ (ergosterol) is the precursor of vitamin D₂. Vitamin D₂ is one of the major forms of vitamin D that is used to fortify foods such as milk and multivitamins.

U.S. Pat. Nos. 5,167,953, 5,194,248 and 5,422,099 disclose topical formulations comprising at least one of lumisterol and tachysterol and derivatives thereof for use in providing vitamin D to individuals throughout the year. The combination of low energy UV photoconversion of lumisterol and tachysterol and derivatives thereof to previtamin D and derivatives during the winter months and high energy UV photoconversion of provitamin D to previtamin D in the summer months provides a method of producing vitamin D in the skin throughout the year.

### SUMMARY OF THE INVENTION

The present invention is related to the discovery that topical formulations comprising provitamin D and derivatives and analogs thereof together with at least one of lumisterol and tachysterol and derivatives and analogs thereof are effective means of providing vitamin D to individuals throughout the year. The present invention utilizes the low energy UV photoconversion of lumisterol and tachysterol and derivatives and analogs thereof to previtamin D and derivatives and analogs thereof during the winter months and high energy UV photoconversion of provitamin D and derivatives and analogs thereof to previtamin D and derivatives and analogs thereof in the summer months as a method of producing vitamin D and derivatives and analogs thereof in the skin throughout the year.

In particular, the invention is directed to a pharmaceutical composition containing an effective amount of

(1) provitamin D or a derivative or analog thereof;

(2) at least one of lumisterol and tachysterol and derivatives and analogs thereof, and

(3) a pharmaceutically effective carrier.

An “effective amount” according to the practice of the invention is optimized according to the time of the year (and the amount of high energy UV radiation received from the sun) and/or the latitude at which the composition is applied to the skin.

In a most preferred embodiment at latitudes between 0° and about 20°, an effective amount corresponds to a weight ratio of provitamin D or derivative or analog thereof to lumisterol and/or tachysterol and derivatives and analogs thereof of about 30:1 to about 1:30, more preferably, about 29:1 to about 1:29, about 28:1 to about 1:28, about 27:1 to about 1:27, about 26:1 to about 1:26, about 25:1 to about 1:25, about 24:1 to about 1:24, about 23:1 to about 1:23, about 22:1 to about 1:22, about 21:1 to about 1:21, about 20:1 to about 1:20, about 19:1 to about 1:19, about 18:1 to about 1:18, about 17:1 to about 1:17, about 16:1 to about 1:16, about 15:1 to about 1:15, about 14:1 to about 1:14, about 13:1 to about 1:13, about 12:1 to about 1:12, about 11:1 to about 1:11, about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1.5:1 to about 1:1.5. Most preferably, the weight ratio of provitamin D or derivative or analog thereof to lumisterol and/or tachysterol and derivatives and analogs thereof is about 1.3:1 during summer, about 1:1 during spring and autumn, and about 1:2 during winter.

In a most preferred embodiment at latitudes between about 20° and about 40°, an effective amount corresponds to a weight ratio of provitamin D or derivative or analog thereof to lumisterol and/or tachysterol and derivatives and analogs thereof of about 30:1 to about 1:200, more preferably, about 29:1 to about 1:150, about 28:1 to about 1:100, about 27:1 to about 1:90, about 26:1 to about 1:80, about 25:1 to about 1:75, about 24:1 to about 1:70, about 23:1 to about 1:60, about 22:1 to about 1:50, about 21:1 to about 1:40, about 20:1 to about 1:30, about 19:1 to about 1:28, about 18:1 to about 1:26, about 17:1 to about 1:24, about 16:1 to about 1:22, about 15:1 to about 1:20, about 14:1 to about 1:19, about 13:1 to about 1:18, about 12:1 to about 1:17, about 11:1 to about 1:16, about 10:1 to about 1:15, about 9:1 to about 1:14, about 8:1 to about 1:13, about 7:1 to about 1:12, about 6:1 to about 1:11, about 5:1 to about 1:10, about 4:1 to about 1:9, about 3:1 to about 1:8, or about 2:1 to about 1:7. Most preferably, the weight ratio of provitamin D or derivative or analog thereof to lumisterol and/or tachysterol and derivatives and analogs thereof is about 1:1 during summer, about 1:2 during spring and autumn, and about 1:6 during winter.

In a most preferred embodiment at latitudes between about 40° and about 60°, an effective amount corresponds to a weight ratio of provitamin D or derivative or analog thereof to lumeristerol and/or tachysterol and derivatives and analogs thereof of about 30:1 to about 1:500, more preferably, about 29:1 to about 1:400, about 28:1 to about 1:300, about 27:1 to about 1:250, about 26:1 to about 1:200, about 25:1 to about 1:175, about 24:1 to about 1:150, about 23:1 to about 1:125, about 22:1 to about 1:100, about 21:1 to about 1:90, about 20:1 to about 1:80, about 19:1 to about 1:70, about 18:1 to about 1:60, about 17:1 to about 1:55, about 16:1 to about 1:50, about 15:1 to about 1:45, about 14:1 to about 1:40, about 13:1 to about 1:38, about 12:1 to about 1:36, about 11:1 to about 1:35, about 10:1 to about 1:34, about 9:1 to about 1:33, about 8:1 to about 1:32, about 7:1 to about 1:31, about 6:1 to about 1:30, about 5:1 to about 1:29, about 4:1 to about 1:28, about 3:1 to about 1:27, or about 2:1 to about 1:26. Most preferably, the weight ratio of provitamin D or derivative or analog thereof to lumisterol and/or tachysterol and derivatives and analogs thereof is about 1:4 during summer, about 1:10 during spring and autumn, and about 1:20 during winter.

In a most preferred embodiment at latitudes between about 60° and 90°, an effective amount corresponds to a weight ratio of provitamin D or derivative or analog thereof to lumeristerol and/or tachysterol and derivatives and analogs thereof of about 25:1 to about 1:4000, more preferably, about 24:1 to about 1:3000, about 23:1 to about 1:2500, about 22:1 to about 1:2000, about 21:1 to about 1:1700, about 20:1 to about 1:1300, about 19:1 to about 1:1000, about 18:1 to about 1:900, about 17:1 to about 1:800, about 16:1 to about 1:700, about 15:1 to about 1:600, about 14:1 to about 1:550, about 13:1 to about 1:500, about 12:1 to about 1:450, about 11:1 to about 1:400, about 10:1 to about 1:380, about 9:1 to about 1:360, about 8:1 to about 1:340, about 7:1 to about 1:320, about 6:1 to about 1:300, about 5:1 to about 1:290, about 4:1 to about 1:280, about 3:1 to about 1:270, about 2.5:1 to about 1:260, about 2:1 to about 1:250, about 1.5:1 to about 1:240, about 1:1 to about 1:230, about 1:1.1 to about 1:220, or about 1:1.3 to about 1:210. Most preferably, the weight ratio of provitamin D or derivative or analog thereof to lumisterol and/or tachysterol and derivatives and analogs thereof is about 1:10 during summer, about 1:50 during spring and autumn, and about 1:200 during winter.

The invention is also directed to a method for providing vitamin D and derivatives and analogs thereof to an individual by administering to the individual a pharmaceutical composition of the invention.

### DESCRIPTION OF THE PREFERRED EMBODIMENTS

The compounds utilized in the present invention are provitamin D, tachysterol, lumisterol and derivatives and analogs thereof, either alone or in combination. The provitamin D, derivatives and analogs thereof include those having the Formula (I):

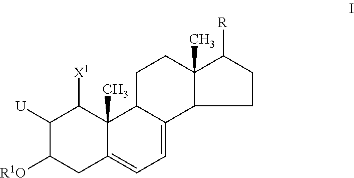

R is a substituted or unsubstituted alkyl, alkenyl or alkynyl group having 1 to 15 C-atoms which may be substituted by one or more hydroxy, halo, lower alkoxy, oxo, oxime, lower alkanoyloxy, aryloxy, aryl, benzoyl, a C₄ lactone, a C₄ lactone substituted by a methyl and a hydroxy group, C₃-C₆ cycloalkyl, or C₃-C₆ cycloalkyl substituted by hydroxy, lower alkyl, or hydroxyloweralkyl;

U is hydrogen, —OH or —O—(C₂-C₄ alkyl)-OH; and

X¹ is selected from the group consisting of hydrogen, —OH and OR¹, or an ester thereof.

Also useful in the practice of the invention are the provitamin D, derivatives and analogs thereof having the Formula (III):

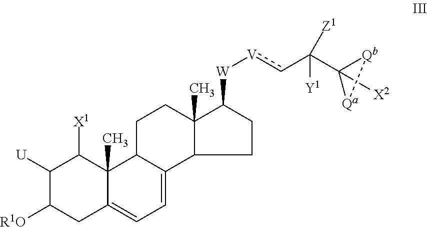

wherein R¹, U and X¹ are defined above;

Q^{a} is CF₃ or CH₂X¹;

Q^{b} is CF₃ or CH₃;

W is CH—CH₃ or O;

V is CH₂ or O;

X² is selected from the group consisting of hydrogen, —OH and OR¹.

Y¹ is hydrogen, F, CH₃, CH₂CH₃ or X¹; and

Z¹ is F, H or X¹;

with the proviso that both W and V are not both O;

“ [symbol] ” is either a single or double bond; and

“ [symbol] ” is either a single bond between Q^{a} and Q^{b} or a hydrogen atom on Q^{a} and Q^{b}, or an ester thereof.

Most preferably, the provitamin D compound is chosen from 1-hydroxyprovitamin D₂, 1-hydroxyprovitamin D₃, 1,24-dihydroxyprovitamin D₂, 1,24-dihydroxyprovitamin D₃, 1,25-dihydroxyprovitamin D₂, 1,25-dihydroxyprovitamin D₃, 24,25-dihydroxyprovitamin D₂, 24,25-dihydroxyprovitamin D₃, 25,26-dihydroxyprovitamin D₂, 25,26-dihydroxyprovitamin D₃, 1,24,25-trihydroxyprovitamin D₂, 1,24,25-trihydroxyprovitamin D₃, 2-β-(3-hydroxypropoxy)-1 alpha,25-dihydroxyprovitamin D₂, and 2-β-(3-hydroxypropoxy)-1 alpha,25-dihydroxyprovitamin D₃, as well as the side chain fluoro derivatives of 1,25-dihydroxyprovitamin D₂, 1,25-dihydroxyprovitamin D₃, 1-hydroxyprovitamin D₂, and 1-hydroxyprovitamin D₃, or esters thereof. Also included are the 20- and 22-oxa provitamin D derivatives including 20-oxa-1α(OH)provitamin D₂, 20-oxa-1α(OH)provitamin D₃, 20-oxa-1α,25 (OH)₂provitamin D₂, 20-oxa-1α,25(OH)₂provitamin D₃, 22-oxa-1α(OH)provitamin D₂, 22-oxa-1α(OH)provitamin D₃, 22-oxa-1α, -25(OH)₂provitamin D₂, and 22-oxa-1α,25(OH)₂provitamin D₃, or esters thereof. Also included within the scope of the present invention are 25,26 cyclopropyl compounds including 1,24-dihydroxy-25,26-dehydroprovitamin D₃ and 1,24-dihydroxy-25,26-dehydroprovitamin D₂ or esters thereof.

The tachysterol derivatives and analogs may have the following Formula (IV):

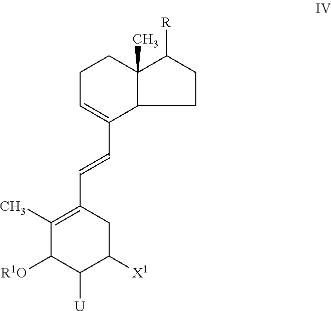

wherein R, R¹, U and X¹ are defined above, or esters thereof.

Preferably, the tachysterol derivatives and analogs have the following Formula (V):

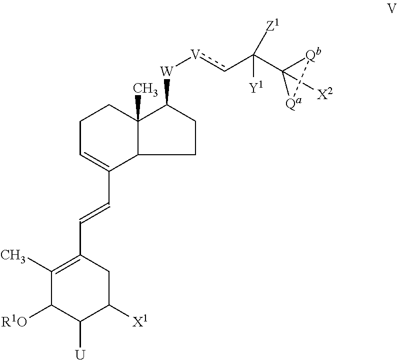

wherein R¹, Q^{a}, Q^{b}, U, V, W, X¹, X², Y¹ and Z¹ are defined above, or esters thereof.

Most preferably, the tachysterol derivatives and analogs are chosen from 1-hydroxytachysterol₂, 1-hydroxytachysterol₃, 1,24-dihydroxytachysterol₂, 1,24-dihydroxytachysterol₃, 1,25-dihydroxytachysterol₂, 1,25-dihydroxytachysterol₃, 24,25-dihydroxytachysterol₂, 24,25-dihydroxytachysterol₃, 25,26-dihydroxytachysterol₂, 25,26-dihydroxytachysterol₃, 1,24,25-trihydroxytachysterol₂, 1,24,25-trihydroxytachysterol₃, 2-β-(3-hydroxypropoxy)-1 alpha,25-dihydroxytachysterol₂, and 2-β-(3-hydroxypropoxy)-1 alpha,25-dihydroxytachysterol₃, as well as the side chain fluoro derivatives of 1,25-dihydroxytachysterol₂, 1,25-dihydroxytachysterol₃, 1-hydroxytachysterol₂, and 1-hydroxytachysterol₃ or esters thereof. Also included are the 20- and 22-oxa tachysterol derivatives including 20-oxa-1α(OH)tachysterol₂, 20-oxa-1α(OH)tachysterol₃, 20-oxa-1α,25(OH)₂tachysterol₂, 20-oxa-1α,25(OH)₂tachysterol₃, 22-oxa-1α(OH)tachysterol₂, 22-oxa-1α(OH)tachysterol₃, 22-oxa-1α,25(OH)₂tachysterol₂, and 22-oxa-1α,25(OH)₂tachysterol₃ or esters thereof. Also included within the scope of the present invention are 25,26 cyclopropyl compounds including 1,24-dihydroxy-25,26-dehydrotachysterol₃ and 1,24-dihydroxy-25,26-dehydrotachysterol₂ or esters thereof.

The lumisterol derivatives and analogs may have the following Formula (VI):

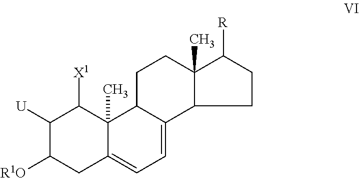

wherein R, R¹, U and X¹ are defined above, or esters thereof.

Preferably, the lumisterol derivatives and analogs have the following Formula (VII)

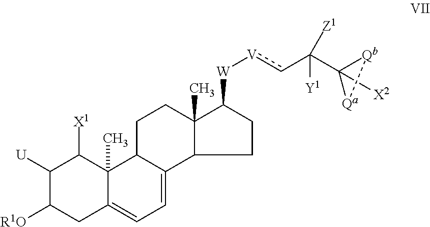

wherein R¹, Q^{a}, Q^{b}, U, V, W, X¹, X², Y¹ and Z¹ are defined above, or esters thereof.

Most preferably, the lumisterol derivatives and analogs are chosen from 1-hydroxylumisterol₂, 1-hydroxylumisterol₃, 1,24-dihydroxyhunisterol₂, 1,24-dihydroxyhunisterol₃, 1,25-dihydroxylumisterol₂, 1,25-dihydroxyhunisterol₃, 24,25-dihydroxylumisterol₂, 24,25-dihydroxylumisterol₃, 25,26-dihydroxyylumisterol₂, 25,26-dihydroxyhunisterol₃, 1,24,25-trihydroxylumisterol₂, 1,24,25-trihydroxylumisterol₃, 2-β-(3-hydroxypropoxy)-1 alpha,25-dihydroxylumisterol₂, and 2-β-(3-hydroxypropoxy)-1 alpha,25-dihydroxylumisterol₃, as well as the side chain fluoro derivatives of 1,25-dihydroxylumisterol₂, 1,25-dihydroxylumisterol₃, 1-hydroxylumisterol₂, and 1-hydroxylumisterol₃, or esters thereof. Also included are the 20- and 22-oxa lumisterol derivatives including 20-oxa-1α(OH)lumisterol₂, 20-oxa-1α(OH)lumisterol₃, 20-oxa-1α,25(OH)₂lumisterol₂, 20-oxa-1α,25(OH)₂lumisterol₃, 22-oxa-1α(OH)lumisterol₂, 22-oxa-1α(OH)lumisterol₃, 22-oxa-1α,25 (OH)₂lumisterol₂, and 22-oxa-1α,-25(OH)₂lumisterol₃ or esters thereof. Also included within the scope of the present invention are 25,26 cyclopropyl compounds including 1,24-dihydroxy-25,26-dehydrolumisterol₃ and 1,24-dihydroxy-25,26-dehydrolumisterol₂, or esters thereof.

Esters include optionally substituted alkanoyl, alkenoyl, aroyl and heteroaroyl esters. Particular alkanoyl esters include C₁₋₂₄ esters such as acetate, propionate, butanoate, isobutanoate, valerate, isovalerate, 2-methylbutanoate, 2,2-dimethylbutanoate, hexanoate, 2-methylvalerate, 3-methylvalerate, 4-methylvalerate, heptanoate, 2-ethylhexanoate, octanoate, nonanoate, decanoate dodecanoate, tetradecanoate, hexadecanoate, eicosanoic, and tetracosanoic esters. Particular alkenyl esters include C16-20 esters such as palmitoleic, oleic, linoleic, linolenic and arachidonic esters. Another example is the substituted alkenoyl ester cinnamoyl. Particular aroyl esters include benzoic, salicylic, toluoyl, anisoyl, and naphthoyl esters. Particular aroyl esters include nicotinoyl, picolinoyl, and furoyl esters.

Useful aryl groups include C₆₋₁₄ aryl, preferably C₆₋₁₀ aryl. Typical C₆₋₁₄ aryl groups include phenyl, naphthyl, 1,2,3,4-tetrahydronaphthyl, phenanthrenyl, anthracenyl, indenyl, azulenyl, biphenyl, biphenylenyl and fluorenyl groups.

Useful heteroaryl groups include thienyl, benzo[b]thienyl, naphtho[2,3-b]thienyl, thianthrenyl, furyl, pyranyl, dihydrobenzofuranyl, benzofuranyl, isobenzofuranyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, b-carbolinyl, phenanthridinyl, acridinyl, perimidinyl, phenanthrolinyl, phenazinyl, thiazolyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, 1,2-benzoisoxazol-3-yl, benzimidazolyl, 2-thio-4-oxo-2,4H-pyrimidyl, 2-oxindolyl and 2-oxobenzimidazolyl.

Optional substituents on the aryl and heteroaryl groups include one or more halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀aryl(C₁-C₆)allyl, C₆-C₁₀ aryl(C₂-C₆)alkenyl, aryl(C₂-C₆)alkynyl, C₁-C₆ hydroxyalkyl, nitro, amino, ureido, cyano, C₁-C₆ acylamino, hydroxy, thiol, C₁-C₆ acyloxy, azido, C₁-C₆ alkoxy or carboxy.

Optional substituents on the alkyl and alkenyl groups include one or more halo, hydroxy, carboxyl, amino, nitro, cyano, C₁-C₆ acylamino, C₁-C₆ acyloxy, C₁-C₆ alkoxy, aryloxy, alkylthio, C₆-C₁₀ aryl, C₄-C₇ cycloalkyl, C₂-C₆ alkenyl, C₂-C₆ alkynyl, C₆-C₁₀ aryl(C₂-C₆)alkenyl, C₆-C₁₀ aryl(C₂-C₆)alkynyl, saturated and unsaturated heterocyclic or heteroaryl.

Particular esters that can be used in the practice of the invention include tachysterol palmitate, lumisterol palmitate, 7-dehydrocholesterol palpitate, tachysterol cinnamate, lumisterol cinnamate, 7-dehydrocholesterol cinnamate, tachysterol oleate, lumisterol oleate, 7-dehydrocholesterol oleate, tachysterol acetate, lumisterol acetate, 7-dehydrocholesterol acetate, tachysterol benzoate, lumisterol benzoate, 7-dehydrocholesterol benzoate, tachysterol linolate, lumisterol linolate and 7-dehydrocholesterol linolate.

These compounds are photoisomers of previtamin D, the precursor of biologically active vitamin D. Tachysterol and lumisterol and derivatives and analogs thereof may be prepared by photoisomerization of provitamin D and derivatives and analogs thereof followed by isolation according to the procedures disclosed by Holick et al., Biochem. 18:1003-1008 (1979), which is fully incorporated by reference herein. Methods for making the corresponding glycosidic and ortho ester glycoside derivatives are taught, for example, by Holick et al., U.S. Pat. Nos. 4,410,515 and 4,521,410, the disclosures of which are fully incorporated by reference herein.

Foremost among the individuals which may be treated with the compositions of the invention are humans, although the invention is not intended to be so limited. Any animal which may benefit from treatment with the compositions of the invention are within the spirit and scope of the present invention.

By using compositions comprising provitamin D, tachysterol, lumisterol and derivatives and analogs thereof in topical compositions according to this invention, it is possible to provide a method which allows individuals living in regions of low energy sunlight to produce year round vitamin D compounds via their skin, thus preventing harmful vitamin D₃ depletion. The compositions of the present invention may be used, therefore, in methods of treating or preventing rickets or osteomalacia due to vitamin D deficiency, and calcium disorders resulting from a lack of vitamin D (a lack of vitamin D leads to deficient intestinal absorption of calcium which results in hypocalcemia), glucocorticoid-induced decrease in calcium absorption, osteoporosis, senile decrease in calcium absorption, hypoparathyroidism, milk fever disease, turkey weak leg disease, decubitus and diabetic foot ulcers, ulcerative keratitis, psoriasis, wound healing, inhibiting scar formation, osteodystrophy due to an acquired or inherited disorder in vitamin D metabolism, and renal osteodystrophy caused by chronic renal failure.

The compounds of the present invention can be administered in any appropriate pharmacological carrier for topical or intravenous administration. The dosage administered will be dependent on the age, health and weight of the recipient, and the nature of the effect desired.

The topical compositions of the invention may be applied so that at least 0.1 microgram, preferably at least about 10 micrograms to about 100 mg of the vitamin D precursors/gm carrier is administered to the skin. A preferred range is between about 1 microgram to about 1 milligram of the vitamin D precursors/gm carrier.

For example, the topical compositions of the present invention may comprise 0.1 μg, 0.2 μg, 0.3 μg, 0.4 μg, 0.5 μg, 0.6 μg, 0.7 μg, 0.8 μg, 0.9 μg, 1 μg, 1.1 μg, 1.2 μg, 1.3 μg, 1.4 μg, 1.5 μg, 2 μg, 2.5 μg, 3 μg, 3.5 μg, 4 μg, 4.5 μg, 5 μg, 6 μg, 7 μg, 8 μg, 9 μg, 10 μg, 15 μg, 20 μg, 25 μg, 30 μg; 35 μg, 40 μg, 45 μg, 50 μg, 60 μg, 70 μg, 80 μg, 90 μg, 100 μg, 200 μg, 300 μg, 400 μg, 500 μg, 600 μg, 700 μg, 800 μg, 900 μg, or 1000 μg of provitamin D, tachysterol, lumisterol, and analogs and derivatives thereof per gram of carrier.

In a most preferred embodiment at latitudes between 0° and about 20°, the composition will comprise, per gram of carrier, between about 0.5 μg and about 600 μg total of provitamin D and/or derivatives or analogs thereof, and between about 0.4 μg and about 500 μg total of lumeristerol and/or tachysterol and/or derivatives or analogs thereof during summer, between about 0.5 μg and about 500 μg total of provitamin D and/or derivatives or analogs thereof, and between about 0.5 μg and about 500 μg total of lumeristerol and/or tachysterol and/or derivatives or analogs thereof during spring and autumn, and between about 0.4 μg and about 500 μg total of provitamin D and/or derivatives or analogs thereof, and between about 0.5 μg and about 500 μg total of lumeristerol and/or tachysterol and/or derivatives or analogs thereof during winter.

In a most preferred embodiment at latitudes between about 20° and about 40°, the composition will comprise, per gram of carrier, between about 0.5 μg and about 500 μg total of provitamin D and/or derivatives or analogs thereof, and between about 0.5 μg and about 500 μg total of lumeristerol and/or tachysterol and/or derivatives or analogs thereof during summer, between about 0.3 μg and about 400 μg total of provitamin D and/or derivatives or analogs thereof, and between about 0.6 μg and about 700 μg total of lumeristerol and/or tachysterol and/or derivatives or analogs thereof during spring and autumn, and between about 0.1 μg and about 200 μg total of provitamin D and/or derivatives or analogs thereof, and between about 0.8 μg and about 900 μg total of lumeristerol and/or tachysterol and/or derivatives or analogs thereof during winter.

In a most preferred embodiment at latitudes between about 40° and about 60°, the composition will comprise, per gram of carrier, between about 0.5 μg and about 500 μg total of provitamin D and/or derivatives or analogs thereof, and between about 0.5 μg and about 500 μg total of lumeristerol and/or tachysterol and/or derivatives or analogs thereof during summer, between about 0.2 μg and about 300 μg total of provitamin D and/or derivatives or analogs thereof, and between about 0.7 μg and about 800 μg total of lumeristerol and/or tachysterol and/or derivatives or analogs thereof during spring and autumn, and between about 0.1 μg and about 40 μg total of provitamin D and/or derivatives or analogs thereof, and between about 0.9 μg and about 1000 μg total of lumeristerol and/or tachysterol and/or derivatives or analogs thereof during winter.

In a most preferred embodiment at latitudes between about 60° and 90°, the composition will comprise, per gram of carrier, between about 0.4 μg and about 400 μg total of provitamin D and/or derivatives or analogs thereof, and between about 0.6 μg and about 600 μg total of lumeristerol and/or tachysterol and/or derivatives or analogs thereof during summer, between about 0.1 μg and about 200 μg total of provitamin D and/or derivatives or analogs thereof, and between about 0.8 μg and about 900 μg total of lumeristerol and/or tachysterol and/or derivatives or analogs thereof during spring and autumn, and between about 0.1 μg and about 5 μg total of provitamin D and/or derivatives or analogs thereof, and between about 1 μg and about 1000 μg total of lumeristerol and/or tachysterol and/or derivatives or analogs thereof during winter.

The compounds can be employed in a pharmacologically inert topical carrier such as one comprising a gel, an ointment or a cream, including such carriers as water, glycerol, alcohol, propylene glycol, fatty alcohols, triglycerides, fatty acid esters or mineral oils. Other possible carriers are liquid petrolatum, isopropylpalmitate, polyethylene glycol ethanol 95%, polyoxyethylene monolaurate 5% in water, sodium lauryl sulfate 5% in water, and the like. Minerals such as anti-oxidants, humectants, viscosity stabilizers and the like may be added, if necessary.

Having now generally described this invention, the same will be understood by reference to an example which is provided herein for purposes of illustration only and is not intending to be limited unless otherwise specified.

### Example 1

This example demonstrates the conversion of 7-DHC and/or T₃ to preD₃ in the presence of winter “natural sunlight” and artificial UVB light which simulates summer sunlight. As can be seen in the following table, compositions comprising only 7-DHC are inefficiently converted to PreD₃ when exposed to winter sunlight. In contrast, compositions comprising increasing amounts of T₃ produce increasing amounts of PreD₃ when exposed to winter sunlight. This suggests that individuals in northern latitudes may apply to their skin compositions comprising predominately T₃ compared to 7-DHC.

When exposed to artificial UVB light, 7-DHC and T₃ are both converted to PreD₃. This suggests that individuals in southern latitudes (year round) and northern latitudes (during the summer) may apply to their skin compositions comprising either 7-DHC and/or T₃.

Conversion of 7-Dehydrocholesterol (7-DHC) and/or Tachysterol (T₃) to Previtamin D₃ (PreD₃)

| | | Length of | Starting | | PreD₃ | |
|---|---|---|---|---|---|---|
| | Exposure | Exposure | Materials (μg) | | Formed | |
| Carrier | Condition | (min.) | 7-DHC | T₃ | (μg) | % Yield |
| Ethanol | Natural | | 10 | 0 | ND | 0 |
| (1 ml) | Sunlight | | 7.5 | 2.5 | 2.1 | 21 |
| | | 90 | 5.0 | 5.0 | 4.1 | 41 |
| | | | 2.5 | 7.5 | 6.1 | 61 |
| | | | 0 | 10 | 7.7 | 77 |
| Cream | Natural | 5 | 0.5 | 0.5 | 0.06 | 6 |
| (1 g) | Sunlight | 10 | 0.5 | 0.5 | 0.17 | 17 |
| | | 20 | 0.5 | 0.5 | 0.25 | 25 |
| | | 60 | 0.5 | 0.5 | 0.19 | 19 |
| | | 5-60 | 1.0 | 0 | ND | 0 |
| | Artificial | 2 | 1.0 | 0 | 0.26 | 26 |
| | UVB | 2 | 0.5 | 0.5 | 0.35 | 35 |
| | | 2 | 0 | 1 | 0.52 | 52 |

Natural Sunlight: 2:00 PM-4:00 PM during Jan. 19-Mar. 1, 2002
Artificial UVB: Hand and Foot UVB box, model 1100A

Having now generally described this invention, it will be apparent to one of ordinary skill in the art that the same can be carried out in a variety of embodiments and variations which are equivalent without affecting the spirit or scope of the invention or any embodiments thereof. All publications, patents, and patent applications are fully incorporated by reference herein.

What is claimed is:

1. A composition comprising a pharmaceutically acceptable carrier and an effective amount of
(a) provitamin D or a derivative or analog thereof;
(b) at least one of lumisterol and tachysterol or analogs and/or esters thereof, and
(c) a pharmaceutically effective carrier;
wherein (a) and (b) are present in amounts effective to provide vitamin D or derivatives or analogs and/or esters thereof to an individual at a particular latitude and/or time of the year.

2. The composition of claim 1, comprising provitamin D₃ and tachysterol₃.

3. The composition of claim 1, wherein said carrier is effective for topical administration.

4. The composition of claim 1, wherein said (a) and (b) are present in an amount of 0.00001 to 10% by weight.

5. A method for providing vitamin D₃ or analog or derivative thereof to an individual which comprises administering to said individual the pharmaceutical composition of claim 1.

6. The method of claim 5, wherein said composition is administered topically.

7. The method of claim 5, further exposing the individual to UV radiation.

8. A method for treating or preventing rickets or osteomalacia due to vitamin D deficiency or a calcium disorder resulting from a lack of vitamin D, glucocorticoid-induced decrease in calcium absorption, osteoporosis, senile decrease in calcium absorption, hypoparathyroidism, milk fever disease, turkey weak leg disease, decubitus and diabetic foot ulcers, ulcerative keratitis, psoriasis, scar formation, osteodystrophy due to an acquired or inherited disorder in vitamin D metabolism, or renal osteodystrophy caused by chronic renal failure in an individual which comprises administering to said individual the pharmaceutical composition of claim 1.

9. The method of claim 8, wherein said composition is administered topically.

10. The method of claim 8, further exposing the individual to UV radiation.
